(19) United States
(12) Patent Application Publication
(10) Pub. No.: US 2007/0128584 A1
(43) Pub. Date: Jun. 7, 2007

(54) Method for isolation of cells

(76) Inventor: Illani Atwater, Santa Barbara, CA (US)

Correspondence Address: FLOYD B. CAROTHERS;CAROTHERS AND CAROTHERS, 445 FORT PITT BOULEVARD, SUITE 500, PITTSBURGH, PA, 15219, US

(21) Appl. No.: 11/291,549

(22) Filed: Dec. 1, 2005

(51) Int. Cl. C12Q 1/00; C12N 5/06

(52) U.S. Cl. 435/4; 435/325

(57) ABSTRACT

A method is disclosed for separating selected cells that have cell membranes which are selectively-permeable to a given solute from other non-selected cells. The combination of cells are exposed to the given solute and thereafter the cells are exposed to low or zero-solute solution to thereby osmotically shock and destroy non-selected cells. Solute examples are ions, sugars, amino acids or other permeant particles. Cells which may be selected for isolation by glucose or a glucose substitute are islets, neurons, incretin secreting cells and cells transfected with the GLUT 2 gene.

### FIELD OF THE INVENTION

The present invention relates to methods for differentiating types of cells or isolating cells. More particularly, the present invention has application to the isolation of islets of Langerhans for transplantation, as well as application to separation of any cells which have a cell membrane that is selectively permeable to a solute from other cells.

### BACKGROUND OF THE INVENTION

Isolation of cells such as islets, neurons, incretin secreting cells and cells transfected with the GLUT 2 gene from other cells under known present day technologies provides disappointing results with low success rates. For example, isolation of islets of Langerhans under present day technology can at maximum yield only about half the islets available in pancreatic tissue. Furthermore, under existing procedures, many of the isolated islets are not viable.

With particular reference to isolation of islets of Langerhans, diabetes, hyperglycemia and impaired glucose tolerance are endocrine disorders characterized by inadequate production or use of insulin, which affects the metabolism of carbohydrates, proteins and lipids resulting in abnormal levels of glucose in the blood. Diabetes is a heterogeneous disease that can be classified into two major groups. One group is type I diabetes (also known as insulin-dependent diabetes, IDDM,juvenile diabetes, or auto-immune diabetes) and type II diabetes (non-insulin dependent diabetes, NIDDM, maturity-onset diabetes).

The cause of the raised glucose levels with insulin-dependent diabetes mellitus (IDDM) is the auto-immune destruction of pancreatic islets of langerhans and, thus, insufficient secretion of the hormone insulin by the pancreas. In the absence of this hormone, the bodies' cells are not able to absorb glucose from the bloodstream causing an accumulation in the blood. Chronically elevated blood glucose damages tissues and organs. IDDM is normally treated with multiple daily insulin injections. The size and timing of insulin injections are determined by measurements of blood glucose and influenced by diet, exercise and stress.

Replenishment of functional glucose-sensing, insulin-secreting pancreatic beta cells through islet transplantation has been a long standing therapeutic target. Recently, a newly developed immune-suppression protocol has dramatically improved the survival of transplanted islets in auto-immune patients. Now the limiting factor in this approach is the availability of an islet source that is safe, reproducible, and abundant. Current methodologies use either human cadaverous material or porcine islets as transplant substrates. Significant problems encountered are the low availability of donor tissue, the variability and low yield of islets obtained via enzymatic dissociation and physical damage that may occur as a result of the isolation process. Separating islets of Langerhans, which represent only about 2% of the pancreatic volume, from the acinar portion of the pancreas is a difficult process. The relatively low success in this procedure has kept islet transplantation an experimental procedure of limited application.

A typical procedure for removal of islets from the pancreas to obtain a tissue suspension of islets and pancreatic tissue is to break down the pancreatic tissue with a digestive enzyme such as collagenase to free the islets. A difficulty with collagenase digestion of the pancreatic tissue to free the islets is that the individual islets are freed at different rates based on their size, distribution, and degree of entrainment or adherence in the tissue. Therefore, during the time collagenase is digesting pancreatic tissue to free the unfreed islets, it is also continuing to act on the islets that have already been freed, thereby breaking up the freed islets into small groups and even individual cells and degrading those cells. The result is that the number of viable islets that are freed by this process is much less than the number of islets in the pancreatic sample that is processed.

Once the islets are separated from the pancreas and suspended in a solution containing islets and partially digested pancreas tissue fragments, there are several techniques for concentrating and purifying them. The most common includes centrifuging the suspension and re-suspending the solution with one containing Ficoll or Percoll, and then centrifuging the tissue fragments through the density gradient so that the islets can be separated from the tissue fragments. Another technique for concentration and purification is the use of filtering, either alone or in combination with centrifuging. Another method partially concentrates the islets by using gravity sedimentation of islets through an inclined channel with a collection well at the bottom. The partially concentrated islets can then be further concentrated with a minimum of ordinary centrifugation or filtering or other processes known in the art.

The separation was first made possible by Lacy's development of a method for differential digestion by collagenase in rats, and this has remained the method used until today. For human islet isolation, a specially purified collagenase is used, Liberase, which is very expensive, costing over $2000 U.S. for each isolation procedure. Ricordi modified the method for use in human pancreas tissue, which is enormously variable in texture, in order to maximize the number of islets separated from each pancreas. His method is known as the semi-automated method, and still requires a very expert team for successful isolation. The time of exposure to Liberase is different for each pancreas, and has to be monitored minute to minute. Nevertheless, only about half of the pancreases processed by an expert team yield enough islets to be transplanted, and, since many of the isolated islets are not viable, most patients require more than one islet preparation to be free of insulin injections.

### SUMMARY OF THE INVENTION

The method of the present invention utilizes differential osmotic shock for isolating not only islets for transplantation, but other cells, such as neurons, incretin secreting cells and cells transfected with the GLUT 2 gene, or any other such cells which have cell membranes which are selectively-permeable to a given solute. The method is based upon the use of high concentrations of solute followed by substitution with low or zero-solute to differentially destroy non-selected cells by osmotic shock. The method yields higher numbers of viable cells, the separation can be carried out in the cold, and the process is much faster and much cheaper than traditional methods. Furthermore, the procedure is a physical process and therefore does not depend upon size and texture of the individual pancreas. The method of the present invention can therefore be fully automated such as by using a machine in the situation for isolating islets of Langerhans wherein the machine is designed to receive the pancreas at one end and deliver the islets at the other.

The present invention provides a method of separating selected cells that have cell membranes which are selectively-permeable to a given solute from other non-selected cells. The method is comprised of exposing the combination of cells to the given.solute and thereafter exposing the combination of cells to a low or zero-solute solution to thereby osmotically shock and destroy non-selected cells. Such solutes, depending upon the cells being isolated, may be ions, sugars, amino acids, or other permeant particles. Typical cells which might be so isolated by the method of the present invention which uses glucose as the differentially permeable solute, are islets, neurons, incretins secreting cells and cells transfected with the GLUT 2 gene. Thus, some non-metabolizable glucose substitutes which are also transported by GLUT 2 could also be used, such as 3-O-methyl-glucose or glucosamine. Also, mannuheptalose can be used to allow glucose to be transported but not metabolized in the islet cells. The method may be carried out in the cold or at room temperature, reducing warm ischemia time, and the osmotically destroyed cells are removed by gentle centrifugation and replacing the solution.

With particular reference to the isolation of islets of Langerhans from a pancreas, the method of the present invention comprises first mincing the pancreas, then exposing it to a high-glucose solution, and after allowing a period of time for accommodation of the acinar cells to the hypertonic solution, replacing the high-glucose solution with a low or zero-glucose solution, thereby osmotically bursting the acinar cells of the pancreas. The treated pancreas tissue suspension is then centrifuged and washed to remove dead acinar cells and cell contents. The islets of Langerhans thus isolated are thereafter placed in culture.

Normally, the high glucose solution is slowly injected into the duct of the pancreas over a period of approximately five minutes. Alternatively, the pancreas may be minced in the high glucose solution. After injecting or mincing, it is further desirable to immerse the pancreas tissue in the same high-glucose solution for a time period, such as approximately twenty minutes. A typical time period for thereafter immersing the pancreas in a low or zero-glucose solution is approximately fifteen minutes. After the mincing and selective osmotic shocking, the pancreas may thereafter also be gently shaken in a cold solution, for example, for an approximate time often to fifteen minutes. Centrifuging and washing may be carried out multiple times, for example several times.

The isolated islets in culture may be maintained at 37° C. in a 5% CO2 incubator. It is also desirable to test the viability of an aliquot of the islets by culturing them in RPMI 1640 with 9 mM glucose supplemented with 15% of FBS, or in Hank's solutions supplemented with albumin, first in 5 mm glucose, then in 15 mm glucose to stimulate secretion.

The solutions used are typically Hank's solutions, Krebs' solutions and physiological solutions.

### DETAILED DESCRIPTION OF PREFERRED EMBODIMENTS

Although the method of the present invention is applicable to the isolation of any cells which have cell membranes that are selectively-permeable to a given solute from other non-selected cells, for the purpose of example, application of the present invention will be discussed in relation to isolation of islets of Langerhans. However, it must be understood that the method of the present invention is also applicable to the isolation of any other cells which also have cell membranes which are selectively-permeable to a given solute.

In this example, the pancreas is extracted from the donor (anesthetized pig or human cadaveric donor) in the traditional fashion, after being perfused with cold preservation solution. It is transported to the islet isolation laboratory in cold solution and cleaned of adhering tissues and fat. The duct is canulated and a modified high-glucose Hank's solution is injected slowly, over a period of five minutes, to inflate the acinar portion of the pancreas. The pancreas is then left for twenty minutes immersed in the same high-glucose solution on ice, or at room temperature. Then, the solution is replaced by a zero-glucose Hank's solution for a further fifteen minutes. The pancreas is minced and after a gentle shaking period often to fifteen minutes in the cold, the mixture is centrifuged and washed several times to remove the dead acinar cells and cell contents. Finally, the islets are placed in culture at 37° C. in a 5% CO₂ incubator until transplant.

To test for viability, islets are cultured in RPMI 1640 medium, or another appropriate culture medium, with approximately 9 mM glucose and supplemented with 15% FBS or human albumin. Insulin secretion is measured in aliquots of the culture medium every 24 hours. After one and after seven days, the islets are tested for response to a glucose challenge. The medium is replaced for one with low glucose (3 mM) for two hours, an aliquot taken and then replaced again for one with 20 mM glucose. After two hours, a second aliquot is taken and the islets are returned to the usual culture medium. Aliquots are frozen until measured for insin, in duplicate, by the Imulite elisa system. By comparing the two aliquots, secretion in response to glucose can be assessed and the viability of the islets van be determined.

The zero-glucose Hank's solution, in mM, is 120 NaCl, 4 KCl, 25 NaHCO₃, 3.5 NaPO₄, 1.2 CaCl₂, 1.2 MgCl₂, 10 HEPES, pH 7.4 (37° C.) and 7.2 (4° C.). The high-glucose Hank's solution is the same as above with 600 mM glucose added. Any physiological solution with and without high concentrations of glucose may be used.

In the cold, glucose metabolism is severely inhibited in the islet cells. At room temperature, the cells may be protected by addition of mannoheptulose, a selective inhibitor of glucose phosphorylation (first step in glucose metabolism). Thus the glucose would be allowed to accumulate more quickly in the cytoplasm to balance the glucose concentration in the medium.

Other applications of the method of the present invention are described and discussed hereinafter.

Osmosis is the flow of solvent through a semi-permeable membrane to equalize the concentration of solutes on either side. In the case of living cells, water is the solvent. Solutes can be ions, sugars, amino acids, or other permeant particles. Thus, this process can be used to separate any type of cell from others, if the cell membrane is selectively permeable to some solute. In the case of the islet cells, the membrane contains a glucose transporter, GLUT 2, which rapidly (within seconds) transports glucose across the membrane to equalize the concentration on either side, thus avoiding a major movement of water. Most cells in the body are relatively impermeable to glucose, except in the presence of insulin (they have glucose transporters, GLUT 4, that are inserted into the membrane after activation of the insulin receptor). Thus, upon application of high glucose, the other cells shrink rapidly. Most cells have many adaptation methods for osmotic shock (Na-Cl exchange, for example), and so over the next 15 minutes or so, the cells gradually return to their normal volume. When they are then exposed to the low or zero-glucose solution, the reverse occurs and the cells swell. If the shock is sufficient, they burst. In most cases, the osmotic shock is lethal, if not immediately, certainly eventually. Any remaining cells die in culture within 24 hours.

Brain neurons have a glucose transporter which is also independent of insulin, GLUT 1. Thus the method can also be used to separate neurons from surrounding cells. The incretin secreting cells of the small intestine have non-insulin dependent glucose transporters and can be separated in this way also. Cells transfected with, and expressing, the GLUT 2 gene (such as artificial beta-cells) can easily be cloned by killing the non-transfected cells with a glucose osmotic shock in accordance with the teachings of the present invention.

In more general terms, with the use of the method of the present invention, any cells semi-permeable to a given solute (a particular amino acid, for example) can be selected from other cells by osmotic shock using solutions high in that solute.

I claim:

1. A method of separating selected cells that have cell membranes which are selectively-permeable to a given solute from other non-selected cells, the method comprising:
exposing the combination of cells to said given solute, and
thereafter exposing the combination of cells to a low or zero-solute solution to thereby osmotically shock, swell and destroy non-selected cells.

2. The method of claim 1, wherein said solutes are selected from the group consisting of ions, sugars, amino acids and other permeant particles.

3. The method of claim 2, wherein said selected cells include cells selected from the group consisting of islets, neurons, incretin secreting cells and cells transfected with the GLUT 2 gene.

4. The method of claim 1, wherein the method is carried out in the cold.

5. The method of claim 1, including the step of removing the destroyed cells.

6. A method for isolating islets of Langerhans from a pancreas, said method comprising:
injecting a modified high-glucose solution into the pancreas to inflate the acinar portion of the pancreas;
after a period time replacing the high-glucose solution with a low or zero-glucose solution for thereby osmotically shocking and swelling acinar cells of the pancreas to destroy acinar tissue;
mincing the treated pancreas;
centrifuging and washing the minced pancreas for thereby removing dead acinar cells and cell contents to leave islets of Langerhans; and
placing the islets in culture.

7. The method of claim 6, wherein the step of injecting includes slowly injecting the solution into the duct of the pancreas.

8. The method of claim 7, wherein the step of injecting is carried out over a period of approximately five minutes.

9. The method of claim 6, after the step of injecting, immersing the pancreas in the same high-glucose solution.

10. The method of claim 9, wherein said pancreas is immersed for approximately twenty minutes.

11. The method of claim 10, wherein after the step of replacing the high-glucose solution with low or zero-glucose solution, exposure of the cells to the low or zero-glucose is carried out for a period of approximately fifteen minutes.

12. The method of claim 6, after the step of mincing, applying a gentle shaking to the minced pancreas in applied cold.

13. The method of claim 12, wherein the step of gentle shaking is carried out for a period of approximately ten to fifteen minutes.

14. The method of claim 6, when the step of centrifuging and washing is carried out multiple times.

15. The method of claim 14, wherein the step of centrifuging and washing is carried out several times.

16. The method of claim 6, wherein the culture is maintained at 37° C. in a 5% CO2 incubator.

17. The method of claim 6, including the step of testing the viability of said islets by culturing said islets in RPMI 1640 with 9 mM glucose supplemented with 15% of FBS.

18. The method of claim 6, wherein the method is carried out in the cold.

19. The method of claim 6, wherein said solutions are selected from the group consisting of Hank's solutions, Krebs' solutions and physiological solutions.

20. The method of claim 6, wherein said islets are tested for viability.
